(19) United States
(12) Patent Application Publication
(10) Pub. No.: US 2025/0325271 A1
(43) Pub. Date: Oct. 23, 2025

(54) BRAIDED OCCLUSION DEVICE HAVING AN INNER COMPRESSION SPRING

(71) Applicant: DEPUY SYNTHES PRODUCTS, INC., Raynham, MA (US)

(72) Inventors: Lacey GOROCHOW, Miami, FL (US); Daniel SOLAUN, Miami, FL (US); Pedro D. PEDROSO, Parkland, FL (US)

(73) Assignee: DEPUY SYNTHES PRODUCTS, INC., Raynham, MA (US)

(21) Appl. No.: 18/641,766

(22) Filed: Apr. 22, 2024

(51) Int. Cl. A61B 17/12; A61B 17/00

(52) U.S. Cl. CPC A61B 17/12113; A61B 17/00234; A61B 17/12031; A61B 17/1214; A61B 17/12177; A61B 2017/00238; A61B 2017/00292; A61B 2017/00526; A61B 2017/00867; A61B 2017/1205

(57) ABSTRACT

An intravascular device that includes a delivery sheath, a shaped braid comprising a distal braid layer and a proximal braid layer, and an inner member comprising a proximal end and a distal end, the inner member disposed between the proximal braid layer and the distal braid layer of the shaped braid. The intravascular device further includes a first configuration, the shaped braid and the inner member having a first length, and a second configuration, the shaped braid and the inner member having a second length wherein the first length is greater than the second length.

The present invention generally relates to devices to be used as medical instruments, and more particularly, to intravascular devices intended for occlusion procedures.

### BACKGROUND

Several different designs for intravascular devices may be used during occlusion procedures. Regardless of the design utilized, the intravascular device will need to perform the general functions of navigating through a patient's vasculature to a target aneurysm, deploying the device in order occlude the opening of the aneurysm, and being able to transform between the two configurations. In order for the intravascular devices to efficiently occlude aneurysms, the device needs to be able to fully occlude the opening and achieve flow stasis. Additionally, aneurysms can range in size and shape and the intravascular devices need to be able to adequately occlude the neck of the aneurysms and fit within the opening appropriately.

Current methods for addressing a range of aneurysms that vary in size and shape is the use of multiple intravascular devices, such as, for example, embolic coils, in order to cover the full range and achieve full occlusion. Unfortunately, this can lead to increased costs for procedure and a larger demand on manufacturing in order to produce multiple occlusion devices that range in size.

As a result, there remains a need for improved intravascular device designs that refine current methods for occluding aneurysms that may range in size and shape. The presently disclosed designs are aimed at providing an improved intravascular device design to allow the device to cover a range of aneurysm sizes and fully occlude aneurysms.

### SUMMARY

In some examples, an intravascular device for occlusion is disclosed. The intravascular device can include a delivery sheath, a shaped braid comprising a distal braid layer and a proximal braid layer, and an inner member having a proximal end and a distal end. In some examples, the proximal end of the inner member can be connected to the proximal braid layer and the distal end of the inner member can be connected to the distal braid layer, the inner member being disposed between the distal braid layer and the proximal braid layer of the shaped braid. In some examples the intravascular device can further include a first configuration having the shaped braid and the inner member disposed inside the delivery sheath, wherein, in the first configuration, the shaped braid and the inner member each have a first length. In some examples, the intravascular device can include a second configuration having the shaped braid and the inner member exit the delivery sheath and are unrestrained, wherein, in the second configuration, the shaped braid and the inner member each have a second length; wherein the first length is greater than the second length.

In some examples, a method of manufacture for an intravascular device is disclosed. The method can include shaping a braid comprising a distal braid layer and a proximal braid layer to, when disposed within a delivery sheath of the intravascular device, have an elongated shape comprising a first length and, when unrestrained, have a disc like shape comprising a second length such that the first length is greater than the second length; heat-setting an inner member to, when disposed within the delivery sheath of the intravascular device, have an elongated shape comprising the first length of the shaped braid, and, when unrestrained, compress to a spring shape comprising the second length of the shaped braid; connecting a distal end of the inner member to the distal braid layer and a proximal end of the inner member to the proximal braid layer; elongating the shaped braid and the inner member; and disposing the shaped braid and the inner member within the delivery sheath the intravascular device.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A illustrates an example intravascular device in a second configuration according to aspects of the present invention;

FIG. 1B illustrates a side view of the example intravascular device of FIG. 1A in a first configuration according to aspects of the present invention;

FIG. 2A illustrates an example embodiment of the intravascular device including a distal coil according to the aspects of the present invention;

FIG. 2B illustrates a side view of the example embodiment of FIG. 2A in a first configuration according to the aspects of the present invention;

FIG. 3 illustrates a view of the intravascular device of FIGS. 2A-2B disposed within an aneurysm of a vessel according to aspects of the present invention; and

FIG. 4 is a flow-diagram illustrating a method of manufacture for an intravascular device according to aspects of the present invention.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

It must also be noted that, as used in the specification and the appended claims, the term “unrestrained” is to be interpreted as meaning “unrestrained by the delivery sheath” in the current context. Further, the term “free air” is similar to the term “unrestrained” and in that both can be interpreted to mean that the specific components are now in “free air” and “unrestrained by the delivery sheath”.

Accessing cerebral, coronary, and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

A common theme across many of the disclosed designs is an intravascular device that includes a shaped braid comprising a distal and proximal braid layer and an inner member disposed therebetween. The intravascular device further comprises a distal coil disposed at a distal end of the deice and connected to a distal end of the distal braid layer. The shaped braid and inner member of the designs disclosed are desirably made from a material capable of recovering its shape automatically once released from a highly strained delivery configuration. A superelastic material such as Nitinol or an alloy of similar properties is particularly suitable. This device can be any of a huge range of shapes as disclosed herein and may be rendered visible under fluoroscopy through the addition of alloying elements such as platinum or through a variety of other coatings or marker bands.

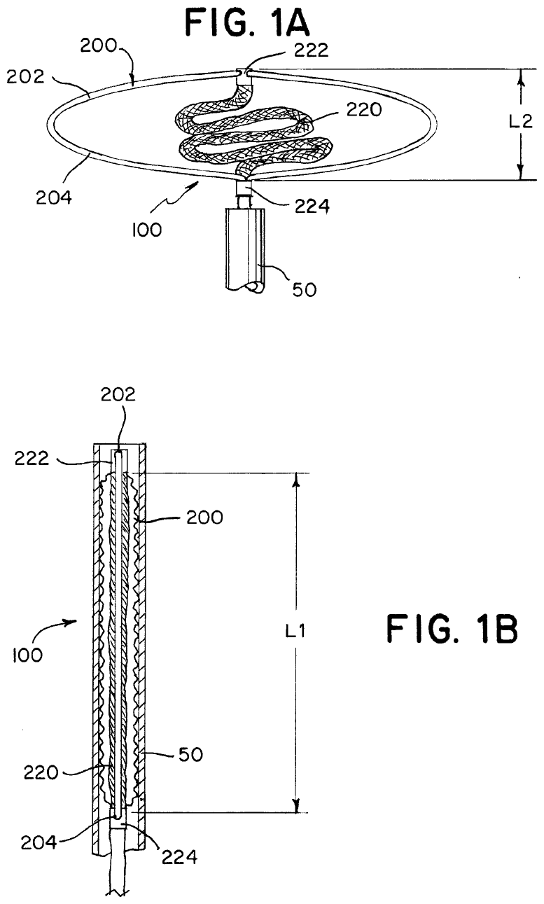

FIGS. 1A and 1B are illustrations of an exemplary intravascular device 100. In some examples, the intravascular device 100 can include a delivery sheath 50, a shaped braid 200 having a distal braid layer 202 and a proximal braid layer 204, and an inner member 220. In some examples, the inner member 220 can be disposed between the distal braid layer 202 and the proximal braid layer 204 of the shaped braid 200 due to a distal end 222 of the inner member 220 connected to the distal braid layer 202 and a proximal end 224 of the inner member 220 connected to the proximal braid layer 204.

In some examples, the intravascular device 100 can include a first configuration, or “delivery configuration”, wherein the intravascular device 100 is configured for delivery to a target site and navigation through the vasculature of a patient. The intravascular device 100 can further include a second configuration, or “occlusion configuration”, wherein the intravascular device 100 is configured for occlusion of an aneurysm.

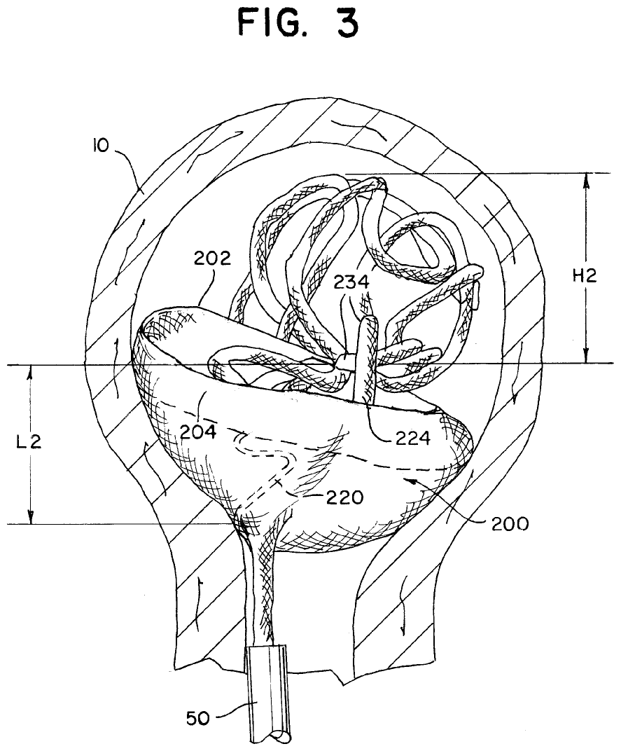

As can be seen in FIG. 1A, when the intravascular device 100 is in the occlusion configuration, the intravascular device 100 can include having both the shaped braid 200 and the inner member 220 exit the delivery sheath 50 and be unrestrained by the delivery sheath 50. In some examples, when the intravascular device 100 is in the occlusion configuration, the inner member 220 can comprise a spring shape such that the inner member 220 compresses the shaped braid 200 by drawing the distal braid layer 202 and the proximal braid layer 204 of the shaped braid 200 towards each other. As a result, the shaped braid 200 is compressed into a disc shape, allowing the intravascular device 100 to occlude an opening in a procedure. This application will be illustrated further in FIG. 3. In some examples, the inner member 220 can comprise Nitinol or any other shape-memory alloy. Further, the inner member 220 can comprise a radiopaque material such that the inner member 220 is visible during fluoroscopy. This can be achieved by coating the inner member 220 in a radiopaque material, the inner member 220 comprising a drawn-filled tube wire of radiopaque material, or any other appropriate method as understood by a person skilled in the pertinent art.

As can be seen in FIG. 1B, when the intravascular device 100 is in the delivery configuration, both the shaped braid 200 and the inner member 220 can comprise a first length L1 and can include an elongated shape such that both can be disposed within the delivery sheath 50 of the intravascular device 100. Further, when the intravascular device 100 is in the occlusion configuration, both the shaped braid 200 and the inner member 220 can comprise a second length L2 such that the first length L1 is greater than the second length L2.

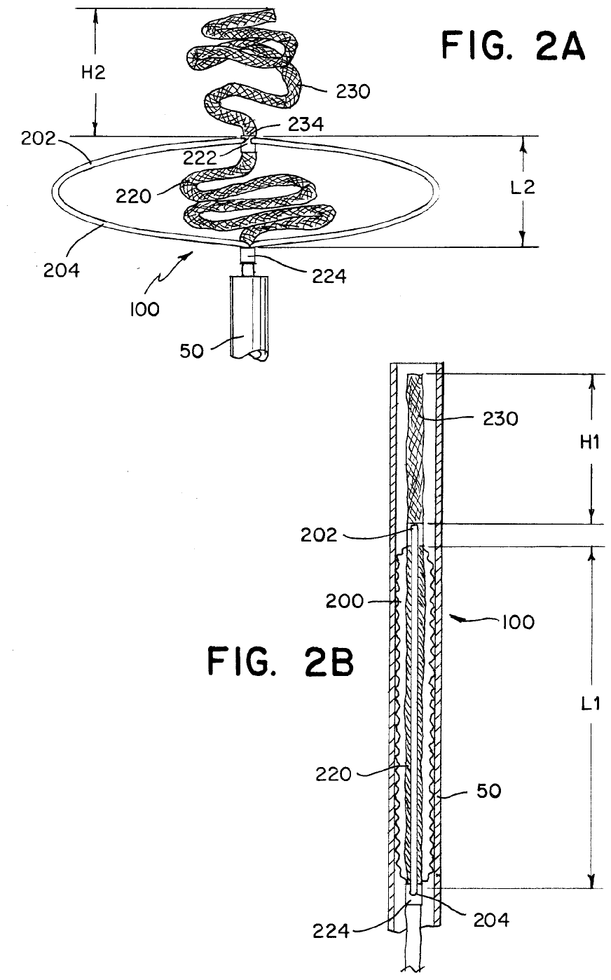

Now referencing FIGS. 2A-2B, which illustrate an example intravascular device 100 similar to the device illustrated in FIGS. 1A and 1B, and further comprising a distal coil 230 disposed at a distal end of the intravascular device 100. FIG. 2A illustrates the intravascular device 100 in an occlusion configuration and FIG. 2B illustrates the intravascular device 100 in a delivery configuration.

In some examples, the intravascular device can include a delivery sheath 50, a shaped braid 200 having a distal braid layer 202 and a proximal braid layer 204, and an inner member 220. In some examples, the inner member 220 extends from the distal braid layer 202 to the proximal braid layer 204 of the shaped braid 200. A distal end 222 of the inner member 220 can be connected to the distal braid layer 202 and a proximal end 224 of the inner member 220 can be connected to the proximal braid layer 204. The intravascular device 100 can further include a distal coil 230 having a proximal end 234 connected to the distal braid layer 202 of the shaped braid 200.

As shown in FIG. 2B, when the intravascular device 100 is in the delivery configuration, the intravascular device 100 can include having each of the shaped braid 200, the inner member 220, and the distal coil 230 in a respective elongated shape such that they can be disposed within the delivery sheath 50 of the intravascular device 100. In some examples, both the shaped braid 200 and the inner member 220 can comprise a first length L1 and the distal coil 230 can comprise a first height H1. As shown in FIG. 2A, when the intravascular device 100 is in the occlusion configuration, the shaped braid 200, inner member 220, and the distal coil 230 exit the delivery sheath 50 and are now exposed to free air. The inner member 220 can compress into a coil spring shape and the shaped braid 200 can compress into a disc like shape.

Further, the distal coil 230 can transform into a spring shape and the inner member 220 can act as a compaction resistant member for the distal coil 230 by opposing the distal coil 230 and preventing excessive force from being applied to the shaped braid 200. In some examples, the distal coil 230 can comprise a platinum wire and can transform into a shape that is complex, helical, helical tapered, or any other appropriate shape as understood by a person skilled in the pertinent art. Additionally, the distal coil 230 can comprise a radiopaque material such that the distal coil 230 is visible during fluoroscopy. This can be achieved by coating the distal coil 230 in a radiopaque material, the distal coil 230 consisting of a drawn-filled tube wire of radiopaque material, or any other appropriate method as understood by a person skilled in the pertinent art.

FIG. 3 illustrates the intravascular device 100 of FIGS. 2A-2B disposed within an aneurysm 10. As can be seen, the intravascular device 100 is in the occlusion configuration such that the shaped braid 200 and the inner member 220 can comprise the length L2 and the distal coil 230 can comprise the second height H2. In some examples, the inner member 220 can transform into a coil spring shape, thereby biasing the distal braid layer 202 and the proximal braid layer 204 of the shaped braid 200 towards each other. As a result, the shaped braid 200 can compress into a disc like shape, thereby occluding the aneurysm 10. Further, the distal coil 230 can transform into a spring shape that even further compresses the shaped braid 200 and further drives the distal braid layer 202 toward the proximal braid layer 204.

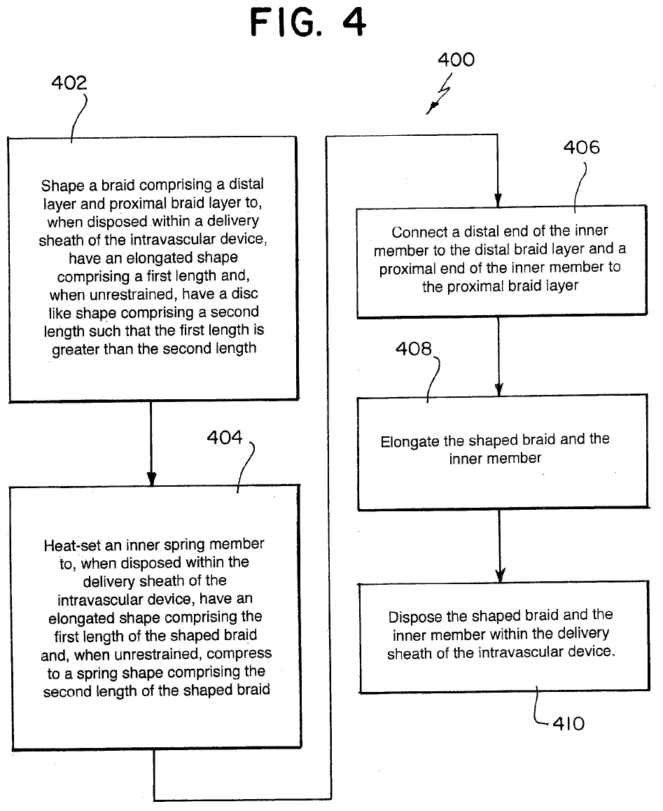

FIG. 4 shows a method 400 of manufacture for intravascular device 100 as disclosed herein. The method 400 steps in FIG. 4 can be implemented by any of the example means described herein or by similar means, as will be appreciated.

At block 402, the method 400 can include shaping a braid 200 comprising a distal braid layer 202 and a proximal braid layer 204 to, when disposed within a delivery sheath 50 of the intravascular device 100, have an elongated shape comprising a first length L1 and, when unrestrained, have a disc like shape comprising a second length L2 such that the first length L1 is greater than second length L2.

At block 404, the method 400 can include heat-setting an inner member 220 to, when disposed within delivery sheath 50 of the intravascular device 100, have an elongated shape comprising the first length L1 of the shaped braid and, when unrestrained, compress to a spring shape comprising the second length L2 of the shaped braid 200.

At block 406, the method 400 can include connecting a distal end 222 of the inner member 220 to the distal braid layer 202 and a proximal end 224 of the inner member 220 to the proximal braid layer 204.

In some examples, before connecting the inner member 220 to the shaped braid 200, the method 400 can further include configuring the inner member 220 to be radiopaque so that the inner member 220 is visible during fluoroscopy.

At block 408, elongating the shaped braid 200 and the inner member 220.

At block 410, disposing the shaped braid 200 and the inner member 220 within the delivery sheath 50 of the intravascular device 100.

In some examples, the method 400 can further include the steps of heat-setting a distal coil 230, when disposed within the delivery sheath 50 of the intravascular device 100, have an elongated shape comprising a first height H1 and, when unrestrained, have a coil shape comprising a second height H2 such that the first height H1 of the elongated shape is greater than the second height H2 of the coil shape; and connecting a proximal end 234 of the distal coil 230 to the distal braid layer 202 of the shaped braid 200. The distal coil 230 can comprise a pattern that is complex, helical, helical tapered shape, or any other desired shape appropriate for the procedure.

In some examples, the distal coil 230 can be configured to, when unrestrained by the delivery sheath 50, compress the inner member 220 by drawing the distal braid layer 202 toward the proximal braid layer 204. The inner member 220 can, when the intravascular device is in the second configuration, act as compaction resistance for the shaped braid 200 through the inner member 220 opposing the distal coil 230 that is compressing into the spring shape.

In some examples, the shaped braid 200 and the inner member 220 can comprise Nitinol or any other shape-memory alloy.

Examples of the present disclosure can be implemented by any of the following numbered clauses:

Clause 1: An intravascular device (100) comprising: a delivery sheath (50); a shaped braid (200) comprising a distal braid layer (202) and a proximal braid layer (204); an inner member (220) having a proximal end (224) and a distal end (222), the proximal end (224) of the inner member (220) being connected to the proximal braid layer (204) and the distal end (222) of the inner member (220) being connected to the distal braid layer (202), the inner member (220) being disposed between the distal braid layer (202) and the proximal braid layer (204) of the shaped braid (200); a first configuration having the shaped braid (200) and the inner member disposed inside the delivery sheath (50), wherein, in the first configuration, the shaped braid (200) and the inner member (220) each have a first length (L1); and a second configuration having the shaped braid (200) and the inner member exit the delivery sheath (50) and are unrestrained, wherein, in the second configuration, the shaped braid (200) and the inner member (220) each have a second length (L2); wherein the first length (L1) is greater than the second length (L2).

Clause 2: The intravascular device (100) of clause 1, wherein the inner member (220), when the intravascular device (100) is in the second configuration, is in the shape of a coil spring, the inner member (220) biasing the distal braid layer (202) and the proximal braid layer (204) towards each other.

Clause 3: The intravascular device (100) of clause 1, wherein the shaped braid (200) is configured to, when the intravascular device (100) is in the second configuration, compress into a disc shape with aid in compression provided by the inner member (220) compressing the shaped braid (200) into the disc shape.

Clause 4: The intravascular device (100) of clause 1, wherein the inner member (220) comprises Nitinol.

Clause 5: The intravascular device (100) of clause 1, wherein inner member (220) comprises a radiopaque material that is visible during fluoroscopy.

Clause 6: The intravascular device (100) of clause 1, further comprising a distal coil (230) attached to a distal end (203) of the distal braid layer (202) of the shaped braid (200).

Clause 7: The intravascular device (100) of clause 6, wherein the distal coil (230) is configured to, when the intravascular device (100) is in the first configuration, have an elongated shape comprising a first height (H1).

Clause 8: The intravascular device (100) of clause 6, wherein the distal coil (230) is configured to, when the intravascular device (100) is in the second configuration, transform into a coil shape comprising a second height (H2) such that the first height (H1) of the first configuration is greater than second height (H2) of the second configuration.

Clause 9: The intravascular device (100) of clause 8, wherein the distal coil (230), when the intravascular device (100) is in the second configuration, can comprise a complex, helical, or helical tapered shape such that the distal coil (230) further compresses the distal layer of shaped braid (200) toward the proximal layer of the shaped braid (200).

Clause 10: The intravascular device (100) of clause 8, wherein the inner member (220), when in the second configuration, is configured to act as a compaction resistant member for the shaped braid (200) by opposing the distal coil (230) when the inner member (220) transforms into the spring shape and the distal coil (230) compresses into the coil shape.

Clause 11: The intravascular device (100) of clause 6, wherein the distal coil (230) comprises a platinum wire having a coil shape.

Clause 12: The intravascular device (100) of clause 6, wherein the distal coil (230) comprises a radiopaque material that is visible during fluoroscopy.

Clause 13: The intravascular device (100) of clause 1, wherein, when the intravascular device (100) is in the first configuration, the shaped braid (200) and the inner member (220) have an elongated shape such that the shaped braid (200) and the inner member (220) are easily disposed within the delivery sheath (50).

Clause 14: A method (400) for producing a intravascular device (100), the method (400) comprising: shaping a braid comprising a distal braid layer (202) and a proximal braid layer (204) to, when disposed within a delivery sheath (50) of the intravascular device (100), have an elongated shape comprising a first length (L1) and, when unrestrained, have a disc like shape comprising a second length (L2) such that the first length (L1) is greater than the second length (L2); heat-setting an inner member to, when disposed within the delivery sheath (50) of the intravascular device (100), have an elongated shape comprising the first length (L1) of the shaped braid (200) and, when unrestrained, compress to a spring shape comprising the second length (L2) of the shaped braid (200); connecting a first end (222) of the inner member (220) to the distal braid layer (202) and a second end (224) of the inner member (220) to the proximal braid layer (204); elongating the shaped braid (200) and the inner member (220); and disposing the shaped braid (200) and the inner member (220) within the delivery sheath (50) of the intravascular device (100).

Clause 15: The method (400) of clause 13, wherein prior to connecting the inner member (220) to the shaped braid (200), the method (400) further comprises the step of:

configuring the inner member (220) to be radiopaque so that the inner member (220) is visible during fluoroscopy.

Clause 16: The method (400) of clause 13, further comprising the step of: heat-setting a distal coil (230) to, when disposed within the delivery sheath (50) of the intravascular device (100), have an elongated shape comprising a first height (H1) and, when unrestrained, have a coil shape comprising a second height (H2) such that the first height (H1) of the elongated shape is greater than the second height (H2) of the coil shape; and connecting a proximal end (234) of the distal coil (230) to the distal braid layer (202) of the shaped braid (200).

Clause 17: The method (400) of clause 16, wherein the distal coil (230) is configured to, when unrestrained by the delivery sheath, compress the distal braid layer (202) and the proximal braid layer (204) toward each other.

Clause 18: The method (400) of clause 16, wherein the coil shape of the distal coil (230) comprises a pattern that is complex, helical, or helical tapered shape.

Clause 19: The method (400) of clause 16, wherein the inner member (220) is configured to act as a compaction resistant member for the shaped braid (200) by opposing the distal coil (230) when the inner member (220) transforms into the spring shape and the distal coil (230) transforms into the coil shape when unrestrained by the delivery sheath.

Clause 20: The method (400) of clause 13, wherein both the shaped braid (200) and the inner member (220) of the intravascular device (100) are comprised of Nitinol.

Clause 21: An intravascular device (100) comprising: a shaped braid (200) comprising a distal braid layer (202) and a proximal braid layer (204); and an inner member (220) having a proximal end (224) and a distal end (222), the proximal end (224) of the inner member (220) being connected to the proximal braid layer (202) and the distal end (222) of the inner member (220) being connected to the distal braid layer (202), the inner member (220) being disposed between the distal braid layer (202) and the proximal braid layer (204) of the shaped braid (200); wherein the shaped braid (200) and the inner member (220) are movable between a first configuration and a second configuration, such that, in the second configuration, the shaped braid (200) and the inner member (220) are unrestrained.

Clause 22: The intravascular device (100) of clause 21, wherein the inner member (220), when the intravascular device (100) is in the second configuration, is in the shape of a coil spring, the inner member (220) biasing the distal braid layer (202) and the proximal braid layer (204) towards each other.

Clause 23: The intravascular device (100) of clause 21, wherein the shaped braid (200) is configured to, when the intravascular device (100) is in the second configuration, compress into a disc shape with aid in compression provided by the inner member (220) compressing the shaped braid into the disc shape.

Clause 24: The intravascular device (100) of clause 21, wherein the inner member (220) comprises Nitinol.

Clause 25: The intravascular device (100) of clause 21, wherein the inner member (220) is configured to be radiopaque so that the inner member (220) is visible during fluoroscopy.

Clause 26: The intravascular device (100) of clause 21, further comprising a distal coil (230) attached to a distal end of the distal braid layer (202) of the shaped braid (200).

Clause 27: The intravascular device (100) of clause 26, wherein the distal coil (230) is configured to, when the intravascular device (100) is in the first configuration, have an elongated shape comprising a first height (H1).

Clause 28: The intravascular device (100) of clause 26, wherein the distal coil (230) is configured to, when the intravascular device (100) is in the second configuration, transform into a coil shape comprising a second height (H2) such that the first height (H1) of the first configuration is greater than second height (H2) of the second configuration.

Clause 29: The intravascular device (100) of clause 28, wherein the distal coil (230), when the intravascular device (100) is in the second configuration, can comprise a complex, helical, or helical tapered shape such that the distal coil (230) further compresses the distal layer of shaped braid (200) toward the proximal layer of the shaped braid (200).

Clause 30: The intravascular device (100) of clause 28, wherein the inner member (220), when in the second configuration, is configured to act as a compaction resistant member for the shaped braid (200) by opposing the distal coil (230) when the inner member (220) compresses into the spring shape and the distal coil (230) compresses into the coil shape.

Clause 31: The intravascular device (100) of clause 26, wherein the distal coil (230) comprises a platinum wire having a coil shape.

Clause 32: The intravascular device (100) of clause 26, wherein the distal coil (230) comprises a radiopaque material that is visible during fluoroscopy.

The descriptions contained herein are examples of embodiments of the invention and are not intended in any way to limit the scope of the invention. As described herein, the invention contemplates many variations and modifications of braided occlusion devices including shaped braids comprising inner members, distal coils, etc. Modifications and variations apparent to those having skilled in the pertinent art according to the teachings of this disclosure are intended to be within the scope of the claims which follow.

What is claimed is:

1. An intravascular device comprising:
a delivery sheath;
a shaped braid comprising a distal braid layer and a proximal braid layer;
an inner member having a proximal end and a distal end, the proximal end of the inner member being connected to the proximal braid layer and the distal end of the inner member being connected to the distal braid layer, the inner member being disposed between the distal braid layer and the proximal braid layer of the shaped braid;
a first configuration having the shaped braid and the inner member disposed inside the delivery sheath, wherein, in the first configuration, the shaped braid and the inner member each have a first length; and
a second configuration having the shaped braid and the inner member exit the delivery sheath and are unrestrained, wherein, in the second configuration, the shaped braid and the inner member each have a second length; wherein the first length is greater than the second length.

2. The intravascular device of claim 1, wherein the inner member, when the intravascular device is in the second configuration, is in the shape of a coil spring, the inner member biasing the distal braid layer and the proximal braid layer towards each other.

3. The intravascular device of claim 1, wherein the shaped braid is configured to, when the intravascular device is in the second configuration, compress into a disc shape with aid in compression provided by the inner member compressing the shaped braid into the disc shape.

4. The intravascular device of claim 1, wherein the inner member comprises Nitinol.

5. The intravascular device of claim 1, wherein inner member comprises a radiopaque material that is visible during fluoroscopy.

6. The intravascular device of claim 1, further comprising a distal coil attached to a distal end of the distal braid layer of the shaped braid.

7. The intravascular device of claim 6, wherein the distal coil is configured to, when the intravascular device is in the first configuration, have an elongated shape comprising a first height.

8. The intravascular device of claim 6, wherein the distal coil is configured to, when the intravascular device is in the second configuration, transform into a coil shape comprising a second height such that the first height of the first configuration is greater than second height of the second configuration.

9. The intravascular device of claim 8, wherein the distal coil, when the intravascular device is in the second configuration, comprises a complex, helical, or helical tapered shape such that the distal coil further compresses the distal layer of shaped braid toward the proximal layer of the shaped braid.

10. The intravascular device of claim 8, wherein the inner member, when in the second configuration, is configured to act as a compaction resistant member for the shaped braid by opposing the distal coil when the inner member transforms into the spring shape and the distal coil compresses into the coil shape.

11. The intravascular device of claim 6, wherein the distal coil comprises a platinum wire having a coil shape.

12. The intravascular device of claim 6, wherein the distal coil comprises a radiopaque material that is visible during fluoroscopy.

13. The intravascular device of claim 1, wherein, when the intravascular device is in the first configuration, the shaped braid and the inner member have an elongated shape such that the shaped braid and the inner member are easily disposed within the delivery sheath.

14. A method for producing an intravascular device, the method comprising:
shaping a braid comprising a distal braid layer and a proximal braid layer to, when disposed within a delivery sheath of the intravascular device, have an elongated shape comprising a first length and, when unrestrained, have a disc like shape comprising a second length such that the first length is greater than the second length;
heat-setting an inner member to, when disposed within the delivery sheath of the intravascular device, have an elongated shape comprising the first length of the shaped braid and, when unrestrained, compress to a spring shape comprising the second length of the shaped braid;
connecting a distal end of the inner member to the distal braid layer and a proximal end of the inner member to the proximal braid layer;
elongating the shaped braid and the inner member; and
disposing the shaped braid and the inner member within the delivery sheath of the intravascular device.

15. The method of claim 14, wherein prior to connecting the inner member to the shaped braid, the method further comprises the step of:
configuring the inner member to be radiopaque so that the inner member is visible during fluoroscopy.

16. The method of claim 14, further comprising the step of:
heat-setting a distal coil to, when disposed within the delivery sheath of the intravascular device, have an elongated shape comprising a first height and, when unrestrained, have a coil shape comprising a second height such that the first height of the elongated shape is greater than the second height of the coil shape; and
connecting a proximal end of the distal coil to the distal braid layer of the shaped braid.

17. The method of claim 16, wherein the distal coil is configured to, when unrestrained by the delivery sheath, compress the distal braid layer and the proximal braid layer toward each other.

18. The method of claim 16, wherein the coil shape of the distal coil comprises a pattern that is complex, helical, or helical tapered shape.

19. The method of claim 16, wherein the inner member is configured to act as a compaction resistant member for the shaped braid by opposing the distal coil when the inner member transforms into the spring shape and the distal coil transforms into the coil shape when unrestrained by the delivery sheath.

20. The method of claim 14, wherein both the shaped braid and the inner member of the intravascular device are comprised of Nitinol.
